(19) United States
(12) Patent Application Publication
(10) Pub. No.: US 2024/0210574 A1
(43) Pub. Date: Jun. 27, 2024

(54) DIGITAL DETECTOR HAVING A LIGHT GENERATOR FOR OPTICAL ERASURE

(71) Applicant: TRIXELL, MOIRANS (FR)

(72) Inventors: Guillaume BACHER, MOIRANS (FR); Mohamed ZEKHNINI, MOIRANS (FR); Frédéric CIARAMELLA, MOIRANS (FR); Patrick CHARRASSE, MOIRANS (FR); Frédéric MAZEL, MOIRANS (FR)

(21) Appl. No.: 18/544,150

(22) Filed: Dec. 18, 2023

(30) Foreign Application Priority Data
Dec. 21, 2022 (FR) 2214164

(51) Int. Cl. G01T 1/20

(52) U.S. Cl. CPC G01T 1/2002

(57) ABSTRACT

A solid-state digital detector for detecting incident radiation, the detector includes a photosensitive sensor and a light generator, the photosensitive sensor comprising photosensitive elements organized in a matrix, the light generator being intended to optically erase the photosensitive elements, wherein the light generator comprises: a light guide comprising a front face facing the front of the detector, a rear face opposite the front face and at least one side face extending between the front face and the rear face, and at least one light source arranged at the lateral periphery of the light guide, and wherein the light generator is configured such that the at least one light source injects light through at least one of the faces of the light guide, the light guide being configured to distribute light from the at least one light source over the entire matrix of photosensitive elements.

### CROSS-REFERENCE TO RELATED APPLICATION

This application claims priority to foreign French patent application No. FR 2214164, filed on Dec. 21, 2022, the disclosure of which is incorporated by reference in its entirety.

### FIELD OF THE INVENTION

The present invention relates to a solid-state X-ray radiation detector comprising a photosensitive sensor able to be associated with a radiation converter.

### BACKGROUND

Such radiation detectors are described for example in French patent FR 2 803 081, in which a sensor formed of amorphous-silicon photodiodes is associated with a radiation converter.

The photosensitive sensor is generally made from solid-state photosensitive elements arranged in a matrix. The photosensitive elements are made from semiconductor materials, more often than not monocrystalline silicon for CCD or CMOS sensors, or polycrystalline or amorphous silicon. A photosensitive element comprises at least one photodiode, phototransistor or photoresistor. These elements are deposited on a substrate, generally a glass panel.

These elements are not generally directly sensitive to very short wavelength radiation like X-rays or gamma rays. This is why the photosensitive sensor has associated with it a radiation converter, also called a scintillator screen, which comprises a layer of a scintillating substance. This substance has the property, when it is excited by such radiation, of emitting radiation of longer wavelength, for example visible light or light close to the visible, to which the sensor is sensitive. The light emitted by the radiation converter illuminates the photosensitive elements of the sensor, which carry out a photoelectric conversion and deliver electrical signals able to be used by appropriate circuits.

In order to improve the quality of the images that are obtained, it is known to carry out before an image capture what is referred to as an “erasure” step, in which the photosensitive elements are optically erased by way of a flash of light distributed uniformly over all of the photosensitive elements.

At present, the flash of light is obtained by way of a light generator formed of a matrix of light-emitting diodes that is placed on the rear face of the detector. By convention, front face of the detector is the name given to the face exposed to the X-ray radiation, and rear face is the name given to the face opposite the front face. During the flash of light, the matrix of light-emitting diodes emits visible radiation that passes through the glass panel forming the substrate of the photosensitive sensor and is then reflected off faces located upstream of the photosensitive sensor before reaching the photosensitive elements.

In some medical imaging applications in which the detector is in motion during an examination, such as for example in computed tomography, an air gap present between the light generator and the detector may have its thickness changed with the movements of the detector. The variation in this air gap may lead to the formation of a ghost image of the light generator on the images from the detector. Another drawback of light generators produced by way of light-emitting diodes or lamps lies in the thickness of such generators. Attempts have also been made to place, on the rear face of the detector, a layer of organic light-emitting diodes, hereinafter called OLED for short. This type of diode is produced in the form of a layer of luminescent material arranged between two electrodes, at least one of which is transparent to allow the emission of light outside the layer. For OLEDs, it is known to use a transparent electrode made of tin-doped indium oxide, hereinafter called ITO for indium tin oxide. This type of electrode is particularly suitable for OLED layers with small surface areas. Indeed, it has been found that ITO has a high resistivity. For an OLED layer with a large surface area, brightness is not homogeneous. Illumination is greater at the edges than at the centre of the layer. X-ray radiation detectors are necessarily large since it is practically impossible to focus this type of radiation due to its high energy. In medical radiology, digital detectors have been produced the dimensions of which are similar to those of silver films used in the past. Detectors the side dimensions of which exceed 400 mm are commonly encountered. For such dimensions, an OLED layer having an ITO electrode would not be able to be used to produce a homogeneous flash of light. Furthermore, the high time constant values linked to the high resistance and to the high capacitance to be charged/discharged of the OLED result in slow response times that are incompatible with obtaining short flashes of light at a high rate.

### SUMMARY OF THE INVENTION

The invention aims to overcome all or some of the problems cited above by proposing a solid-state digital radiation detector in which a uniformly distributed flash of light makes it possible to reset the pixels. The invention is of great interest for an X-ray radiation detector. It is nevertheless possible to implement the invention for other types of radiation, such as for example gamma radiation.

To this end, the invention relates to a solid-state digital detector for detecting incident radiation, the detector comprising a photosensitive sensor and a light generator, the photosensitive sensor comprising photosensitive elements organized in a matrix, the light generator being intended to optically erase the photosensitive elements, wherein the light generator comprises:
a light guide comprising a front face facing the front of the detector, a rear face opposite the front face and at least one side face extending between the front face and the rear face, and
at least one light source arranged at the lateral periphery of the light guide, and wherein the light generator is configured such that said at least one light source injects light through at least one of the faces of the light guide,
said light guide being configured to distribute light from said at least one light source over the entire matrix of photosensitive elements.

The configuration of the digital detector of the invention advantageously makes it possible to limit the overall size of the light generator. Said at least one light source is thus arranged facing or close to the side faces of the light guide. Since light arrives laterally in the guide, the distance travelled to reach the photosensitive elements is increased, thereby improving the diffuse aspect of light at the photosensitive elements and reducing, or even cancelling out, the distinctiveness of light from each light source. The invention also enables operation of the detector at high frequencies and short light pulses with short on and off times.

The invention also makes it possible to reduce both the number of light sources and the size of the associated printed circuits compared to the prior art. Furthermore, since the surface area of the printed circuits is reduced, this achieves a large reduction in or even an absence of deformation thereof and therefore a reduction in artefacts in the images. This reduction in surface area also makes it possible to reduce the probability of impact in the event of the digital detector being dropped. Replacement of the light sources is also facilitated.

In one embodiment of the invention, the light guide comprises a diffusive texturing in order to achieve a uniform distribution of light across the matrix of photosensitive elements.

According to one embodiment of the invention, the light guide comprises a set of microbubbles expediently arranged inside it in order to achieve a uniform distribution of light across the matrix of photosensitive elements.

According to one embodiment of the invention, the photosensitive sensor comprises a transparent or translucent substrate on which the matrix of photosensitive elements is arranged and wherein the front face of the light guide is arranged against said substrate.

The light guide is notably attached to the substrate of the photosensitive sensor, preferably by adhesive bonding.

According to one embodiment of the invention, the photosensitive sensor comprises a transparent or translucent substrate on which the matrix of photosensitive elements is arranged and wherein said substrate forms the light guide.

According to one embodiment of the invention, said at least one light source is arranged facing at least one of the side faces of the light guide.

According to one embodiment of the invention, the light generator comprises at least one reflection member able to deflect light from said at least one light source towards the light guide.

The reflection member notably comprises at least one dioptre and/or at least one reflector arranged facing one of the faces of the light guide.

According to one embodiment of the invention, the light guide comprises at least two mutually opposing side faces, wherein the light generator comprises at least one lateral reflector arranged against at least one of the side faces of the light guide, and said at least one light source is preferably arranged facing the side face opposite that of the lateral reflector.

According to one embodiment of the invention, the matrix of photosensitive elements is arranged on a transparent or translucent substrate, wherein the light guide is arranged downstream of said substrate and comprises a lateral part offset from that of said substrate, wherein the light generator comprises at least one upper reflector extending against all or part of the upper face of said at least one offset lateral part of the light guide, and wherein said at least one light source is arranged at the periphery of said at least one offset lateral part of the light guide.

### BRIEF DESCRIPTION OF THE DRAWINGS

The invention will be better understood on reading the following description, which is given solely by way of example and with reference to the appended drawings, in which:

FIGS. 1A-1E show five configurations of a digital detector according to the invention. Each configuration is shown in a sectional view.

FIGS. 2A-2B show a sectional view of the digital detector from FIG. 1A according to a first configuration of a light generator. In FIG. 2A, the light generator comprises a light source and, in FIG. 2B, it comprises two mutually opposing light sources.

FIGS. 3A-3B show a sectional view of the digital detector from FIG. 1A according to a second configuration of a light generator. In FIG. 3A, the light generator comprises a light source and, in FIG. 3B, it comprises two mutually opposing light sources.

FIGS. 4A-4B show a sectional view of the digital detector from FIG. 1A according to a third configuration of a light generator. In FIG. 4A, the light generator comprises a light source and, in FIG. 4B, it comprises two mutually opposing light sources.

### DETAILED DESCRIPTION

The following embodiments are examples. Although the description makes reference to one or more embodiments, this does not necessarily mean that each reference relates to the same embodiment, or that the features are applicable only to a single embodiment. Simple features of various embodiments may also be combined and/or interchanged in order to provide other embodiments.

In the present description, it is possible to index certain elements or parameters, such as for example a first reflector or a second reflector, etc. In this case, this is simple indexing for the purpose of differentiating and naming elements, parameters or criteria that are close but not identical. This indexing does not imply any priority of one element, parameter or criterion over another, and such denominations may easily be interchanged without departing from the scope of the present description. This indexing also does not imply any order in time.

A solid-state digital detector 1 according to the invention makes it possible to form an image based on incident radiation to which it is sensitive, such as for example X-ray radiation 100. The detector 10 comprises a scintillator screen 20 for converting the X-ray radiation 100 into visible radiation, and a photosensitive sensor 30 comprising photosensitive elements 31 for converting the visible radiation from the scintillator screen 20 into electrical signals forming the image. The detector additionally comprises a light generator 40 intended to optically erase the photosensitive elements 31 of the sensor 30.

It will be understood that the detector 10 may comprise other components.

The invention may be implemented in a digital detector 1 without a scintillator screen and the sensor 30 of which converts the radiation 100 directly into an electrical signal. To this end, the photosensitive sensor 30 comprises a photoconductor directly sensitive to the incident X-ray radiation 100.

More precisely, during operation of the detector 10, in order to obtain an image, a series of operations is carried out, formed of an image-capturing phase during which the detector 10 is subjected to the X-ray radiation 100, followed by a readout phase during which the electrical signals from each of the photosensitive elements 31 are read, and then by an optical erasure phase during which all of the photosensitive elements 31 are illuminated uniformly by way of the light generator 40, and finally an electrical reset phase of resetting all of the photosensitive elements 31.

By convention, the front face of the detector 10 is defined as the face receiving the X-ray radiation 100 first, the rear face being opposite the front face. The relative positions of the various components of the detector 10 in relation to one another are also defined with respect to the direction of propagation of the X-ray radiation. It will be stated for example that the scintillator screen 20 is located upstream of the photosensitive sensor 30 because the scintillator screen 20 receives the X-ray radiation 100 before the photosensitive sensor 30. In practice, the sensor 30 receives only very little X-ray radiation 100, since the X-ray radiation 100 is almost entirely converted into visible radiation by the scintillator screen 20. The concept of upstream and downstream may also be understood for the direction of propagation of the visible radiation that the scintillator screen 20 emits in the direction of the sensor 30. In the context of the invention, the terms “upper”, “top”, “upstream” and “front” will be taken as synonyms, and the terms “lower”, “downstream”, “bottom” and “rear” will be taken as synonyms.

The scintillator screen 20 is notably in contact with the photosensitive elements 31. The scintillator screen 20 comprises notably a scintillating substance 21 from the family of alkali metal halides or rare-earth oxysulfides. The scintillating substance 21 consists notably of fluorescent crystals such as sodium iodide crystals or else sodium-doped or thallium-doped caesium iodide crystals.

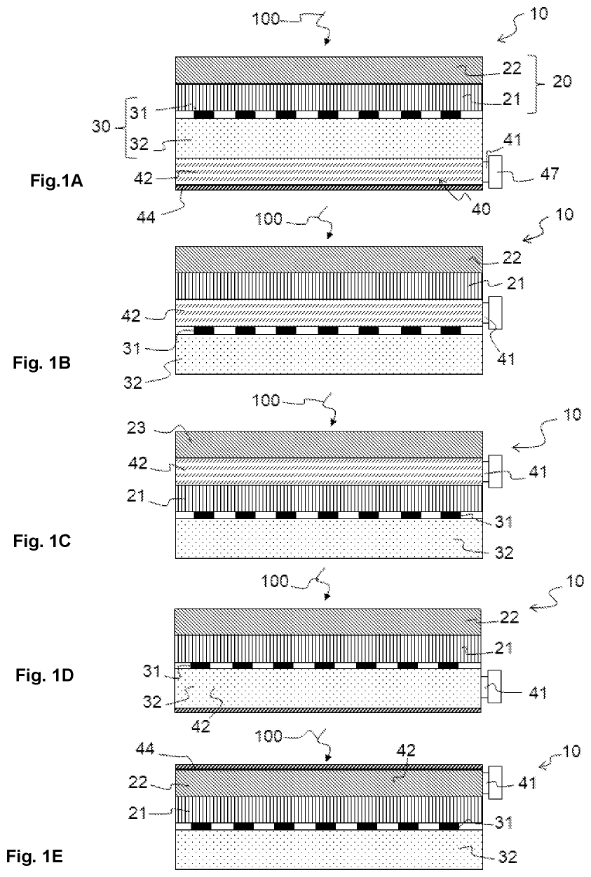

In a first configuration, referred to as added scintillator screen configuration, the scintillating substance 21 is deposited on a support 22 through which the X-ray radiation 100 has to pass before reaching the sensor 30. The assembly is then adhesively bonded to the sensor 30 (FIGS. 1A-1E).

In a second configuration, referred to as direct deposition configuration, the sensor 30 serves as a support for the scintillating substance 21, which is then in direct and close contact with the sensor 30. The scintillating substance 21 is then covered with a protective sheet 23.

In one embodiment, the support 22 or the protective sheet 23 is notably transparent or translucent. Transparent is understood to mean an element that makes it possible to distinguish objects clearly through the thickness thereof. An element may be considered to be transparent when it exhibits light transmission between 80% and 99%. Translucent is understood to mean an element that lets through light in the visible range, but without making it possible to distinguish objects clearly. An element may be considered to be translucent when it exhibits light transmission greater than or equal to 50% and less than 80%.

In order to be transparent or translucent, the support 22 or the protective sheet 23 is notably made of glass or plastic. By way of example, plastics exhibiting good transparency and that may be suitable for this purpose are: polypropylene, polyethylene terephthalate, polymethyl methacrylate, polycarbonate, polyethylene terephthalate, acrylonitrile styrene acrylate, acrylonitrile butadiene styrene, or else amorphous polyolefins such as cyclic olefin copolymers (COCs) or cyclic olefin polymers (COPs). Each of these materials obviously has its own known refractive index. With regard to each of these materials, and more particularly polypropylene, a person skilled in the art knows how to determine therefrom the members corresponding to the transparent materials as defined above.

As an alternative, the support 22 or the protective sheet 23 is opaque and may serve as a reflector for returning the light flux from the light generator 40 in the direction of the photosensitive elements 31. In particular, the support 22 or the protective sheet 23 may be made of aluminium or of titanium.

The support 22 or the protective sheet 23 may comprise notably a diffusive texturing on the lower surface thereof in order to achieve a uniform distribution of light across the matrix of photosensitive elements 31. Said texturing may correspond to a frosted appearance of said surface, to an indentation or to screen-printed dots. Said texturing may notably be obtained through a controlled chemical attack, through mechanical machining or any other technique known to a person skilled in the art, such as localized deposition or laser texturing, for obtaining the desired surface characteristics. Notably again, the support 22 or the protective sheet 23 may comprise a set of microbubbles expediently arranged inside it in order to make it possible to achieve diffusion of incident light. Notably again, the lower surface of the support 22 or of the protective sheet 23 may comprise a set of spots the dimensions and/or number of which increase with the distance from the at least one light source 41 of the generator 40. In particular, the spots are circular. These aspects of the invention are of interest notably when the light guide 42 of the generator 40 is arranged upstream of the support 22 or of the protective sheet 23, or else when the latter form the light guide 42, as will be seen in detail below.

The photosensitive elements 31 are organized in a matrix and are notably arranged on a substrate 32. The photosensitive elements 31 may be made from semiconductor materials; they are notably made of monocrystalline silicon or polycrystalline or amorphous silicon, notably of hydrogenated amorphous silicon. The photosensitive elements 31 may be spaced from one another on the substrate 32 by gaps. Functional components allowing the photosensitive elements 31 to operate may be arranged in the gaps separating said photosensitive elements. The functional components are notably arranged on the substrate 32 as well. These include for example a follower transistor, a readout transistor and a reset transistor associated with each of the photosensitive elements 31. A pixel denotes the assembly formed by a photosensitive element 31 and the associated functional components. Electrical conductors may also circulate in the gaps, making it possible to supply power to, control and read the various photosensitive elements 31 via the functional components.

The photosensitive elements 31 are distributed over all or part of the upper surface of the substrate 32. For the remainder of the description, the region of the upper surface of the substrate 32 comprising the photosensitive elements will be called “image zone”.

The substrate 32 is notably transparent or translucent and has notably a composition chosen from among those listed above for the support 22.

The lower surface of the substrate 32 may notably comprise a diffusive texturing and/or a set of spots as described with reference to the support 22. The substrate 32 may also comprise a set of microbubbles inside it, as described above for the support 22. These aspects of the invention are of interest notably when the light guide 42 of the generator 40 is arranged behind the substrate 32, or else when the substrate 32 serves as a light guide, as will be seen in detail below.

The light generator 40 comprises at least one light source 41 emitting a visible, near-ultraviolet or near-infrared light flux into a light guide 42 configured to distribute light from said at least one light source 41 over all of the photosensitive elements 31. The light guide 42 comprises a front face facing the front of the detector, a rear face opposite the front face and at least one side face extending between the front face and the rear face. “Near-ultraviolet light” and “near-infrared light” are understood in the invention to mean electromagnetic wavelengths in the ultraviolet or in the infrared, respectively, which are close to the visible window, such that the photosensitive elements 31 are sensitive thereto. The at least one light source notably emits in a wavelength range from 0.2 nm to 1 nm.

Said at least one light source 41 is arranged at the lateral periphery of the light guide, that is to say in contact with or close to at least one of the side faces of said light guide 42. This arrangement makes it possible to achieve an optimization of the overall size of the light generator 40 with respect to the thickness of the detector 10. “Close” is understood in the invention to mean that said at least one light source 41 is arranged at a distance close to a side face of the guide 42, allowing it to diffuse a light flux into the light guide 42. Thus, in the invention, the at least one light source 41 is not necessarily arranged facing a side face of the light guide 42; it may be arranged above or below.

The at least one light source 41 is arranged at the periphery of all or part of at least one side face of the light guide 42. All of the light sources 41 are notably arranged at the periphery along one and the same side face of the light guide 42, or along two opposing side faces of the light guide 42.

Said at least one light source 41 is notably a light-emitting diode. Said at least one light source 41 may be associated with a printed circuit board 47 managing the switching-on and switching-off thereof.

The light generator 40 may notably furthermore comprise at least one reflection member 43 able to deflect light from said at least one light source 41 towards the light guide 42. Light from said at least one light source 41 may thus be concentrated in the light guide, returned in the light guide or routed towards the light guide 42, as will be seen in detail below. This last aspect is of particular interest when said at least one light source 41 is arranged close to at least one of the side faces of the light guide 42, without facing it.

The at least one reflection member 43 notably comprises at least one dioptre and/or at least one reflector arranged facing one of the faces of the light guide 42. The reflection member 43 may notably comprise:
at least one lower dioptre/reflector 44 arranged facing the lower surface of the guide 42, and/or
at least one upper dioptre/reflector 45 arranged facing the upper face of the guide 42, and/or
at least one lateral dioptre/reflector 46 arranged facing the or one of the side faces of the guide 42.

The surface of the dioptre or of the reflector 44, 45, 46 may be plane and/or follow the shape of the surface of the guide 42 arranged facing it. As an alternative, the surface of the dioptre or of the reflector 44, 45, 46 may be oblique to the surface that it is facing, this surface corresponding notably to the entry surface. In particular, the oblique dioptre or reflector 44, 45, 46 forms notably an angle, with the surface of the guide 42 that it is facing, of 45°. The dioptre or the reflector 44, 45, 46 is notably in contact with the surface of the guide 42 arranged facing it. The lateral dioptre or reflector 46 also extends notably facing a side face of the sensor 30, notably of the substrate 32, and/or a side face of the scintillator screen 20, notably of the scintillating substance 21. The upper dioptre or reflector 45 is arranged notably on an offset lateral part of the light guide 42, described in detail below. When the dioptre or reflector 44, 45, 46 is oblique, it makes it possible notably to direct the light flux from the at least one light source 41 when said light source is not arranged facing a side face of the guide 42.

The reflector 44, 45, 46 has notably a white reflection surface, making it possible to achieve maximum reflection. The reflector 44, 45, 46 may also be a mirror.

The light generator 40 may be configured such that at least part of the visible light flux arrives transversely to the guide 42, notably by way of said at least one reflection member 43, so as to improve the diffusion of the light flux.

The light guide has one or more entry surfaces through which said light source 41 emits a light flux. The at least one entry surface may correspond to a side face of the guide 42. As an alternative or in addition, the at least one entry surface may correspond to a part of the upper surface and/or to a part of the lower surface of the guide 42.

Furthermore, the light guide 42 has at least one exit surface through which light from said at least one light source 41 leaves. In the invention, said at least one exit surface is arranged at least facing the photosensitive elements 31. Said at least one exit surface corresponds notably to the upper surface and/or to the lower surface of the guide 42. Said at least one exit surface may furthermore correspond to at least one of the side surfaces of the light guide. Said exit surface is notably a continuous surface. When the exit surface is not arranged facing the photosensitive elements 31, it preferably faces a reflection member 43 of the light generator 40, a dioptre or a reflector of the detector 10, allowing said light to be routed or returned to the photosensitive elements 31, in order to maximize the illumination of the photosensitive elements 31.

The light guide 42 is notably a diffusive guide, making it possible to distribute the light flux substantially constantly at any point of the at least one exit surface of the guide 42.

To this end, the light guide 42 may notably comprise a diffusive texturing, a set of microbubbles and/or a set of spots as described with reference to the support 22. Said diffusive texturing and said set of spots are arranged at least on the at least one exit surface of the guide 42. In particular, they are arranged at least on the exit surface facing the photosensitive elements 31 and optionally also facing at least one reflection member 43, notably the lower reflector 44.

The various embodiments in relation to the diffusive aspect of the guide 42 may of course be combined with one another.

The light guide 42 is notably transparent or translucent and has notably a composition chosen from among those listed for the support 22.

The light guide 42 extends at least over the entire surface of the upper and/or lower adjacent element. The guide 42 is notably in the form of a plate the upper surface and the lower surface of which are at least the same as those of the element arranged above or below. The light guide 42 may also be in the form of a multitude of fibres aligned in parallel with one another.

Furthermore, the light guide 42 may have larger dimensions than the upper adjacent element and/or the lower adjacent element. The light guide may thus comprise at least one lateral part that is offset from the periphery of the substrate 32 of the sensor 30 and/or of the scintillator screen 20. This aspect of the invention makes it possible, using an upper dioptre or reflector 45 and/or a lower dioptre or reflector 44, to minimize or even avoid light escaping close to the at least one light source 41 and to concentrate light from the at least one light source 41 inside the guide.

The light guide 42 may be arranged at various levels inside the detector 10. The detector may thus be arranged:
directly downstream of the substrate 32 of the sensor 30,
between the scintillating substance 21 and the photosensitive elements 31,
between the protective sheet 23 and the scintillating substance 21, and
upstream of the support 22 or of the protective sheet 23 of the scintillator screen 20.

The composition of the substrate 32, of the protective sheet 23 and of the support 22 may easily be adapted on the basis of the position of the light guide 42 and of the need to transmit the light flux from the at least one light source 41 or else to return said light flux.

According to one embodiment of the invention, the light guide 42 is in contact with the adjacent element via its exit surface facing the photosensitive elements 31. Said adjacent element may be the substrate 32 of the sensor, the scintillating substance 21 of the scintillator screen, the support 22 of the scintillator screen 20 or the protective sheet 23 of the scintillator screen 20. The absence of an air gap between the light guide 42 and the adjacent element allows more homogeneous diffusion of light in the direction of the photosensitive elements 31. The light guide 42 may be attached by any means to the adjacent element via its exit surface arranged facing the photosensitive elements. This thus achieves an integral structure in which the light guide 42 follows any deformation of the element adjacent to its exit surface facing the photosensitive elements 31. This aspect makes it possible notably to ensure that all of the photosensitive elements 31 receive part of the flux emitted by the at least one light source 41 and that the distance between each photosensitive element 31 and the light guide 42 remains fixed. The fact that local variations in light intensity may occur at one or more photosensitive elements 31 is not problematic in the invention.

This attachment is achieved notably by adhesive bonding. The adhesive bonding may be carried out over all or part of the exit surface facing the photosensitive elements 31. The adhesive bonding may notably be carried out in the region of said exit surface facing the image zone of the sensor 30, or else outside this image zone and then correspond to peripheral adhesive bonding. The adhesive that is used may notably have a refractive index lower than that of the adjacent element, making it possible to achieve better light guidance. This aspect is of interest when the adhesive is present in the region of said exit surface facing the image zone of the sensor 30.

Furthermore, the light guide 42 may also correspond to the substrate 32 of the sensor 30, to the support 22 or to the protective sheet 23 of the scintillator screen 20, and also to the scintillating substance 21 of the scintillator screen 20.

The light generator 40 may also comprise a diffuser 49 arranged between the light guide 42 and the photosensitive elements 31, notably in contact with the exit surface or one of the exit surfaces of the light guide 42. The diffusive aspect of the diffuser 49 is achieved notably via the various embodiments described with reference to the light guide 42, which are applicable mutatis mutandis. The diffuser is made of a transparent or translucent material, as described above.

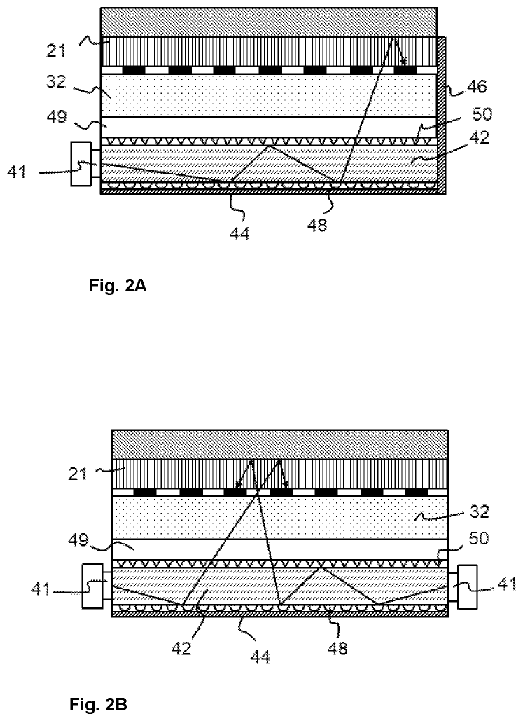

FIGS. 1A to 1E make it possible to illustrate five configurations according to the invention of the arrangement of the light guide 42 with respect to the other components of the detector 10. In these figures, a single light source 41 with its printed circuit have been shown for greater clarity. Other configurations of the light generator 40 are possible, and are illustrated notably in FIGS. 2A-4B.

FIG. 1A shows a first configuration in which the following are found from upstream to downstream of the detector 10: the support 22, the scintillating substance, the photosensitive elements 31, the substrate 32 of the sensor 30, the light guide 41 and a lower reflector 44. Thus, in this configuration, the light guide 41 is arranged on the side of the rear face of the detector 10. In this configuration, the exit surface of the light guide 42 corresponds at least to its upper surface. The flash of light emitted by the light source 41 for the optical erasure of the photosensitive elements 31 thus leaves the guide 42 via its upper surface, passes through the substrate 32, passes between the photosensitive elements 31 and is reflected off faces located upstream of the photosensitive sensor 30 before reaching the photosensitive elements 31 via their upstream face. More precisely, the flash of light may be reflected at the interface between the support 22 and the scintillating substance and/or at the interface between the scintillator screen 20 and the photosensitive sensor 30 off faces where there is a break in refractive index. So as to propagate along the light guide 42, the flash of light is reflected against the lower reflector 44 and may also be reflected off the rear face of the photosensitive elements 31 and/or at the interface between the light guide 42 and the substrate 32 of the sensor 30. The rear surface of the photosensitive elements 31 may thus be able to reflect visible light. This thus increases the possibility of light from the light source 41 reaching all of the photosensitive elements 31. In this case, the support 22 is notably made of an opaque material.

In the configuration that is shown, the substrate 32 should be transparent or translucent so as to let through light emitted by the generator 40.

In this configuration, the light guide 42 is notably in contact with and in particular adhesively bonded to the substrate 32 of the sensor 30 via its upper surface.

The lower reflector 44 is able here to return light leaving via the lower face of the guide 42 towards the guide 42 and notably in the direction of its upper face in order to reach the upper layers and the photosensitive elements 31.

FIG. 1B shows a second configuration in which the following are found from upstream to downstream of the detector 10: the support 22, the scintillating substance 21, the light guide 42 and the sensor 30. In this configuration, the exit surface of the light guide 42 corresponds at least to its lower surface and to its upper surface, the latter allowing photons from the scintillator screen 20 to reach the photosensitive elements 31. The flash of light emitted by the light source 41 thus arrives directly at the photosensitive elements 31 from the lower surface of the light guide 42. There is therefore no need for a reflection member 43 in this configuration. So as to propagate along the light guide 42, the flash of light may be reflected at the interface between the light guide 42 and the scintillator screen 20 and/or at the interface between the scintillating substance 21 and the support 22. In this case, the support 22 is notably made of an opaque material. The light guide 42 may notably be in contact with the photosensitive elements 31. Advantageously, in this configuration, the diffusion of light to the photosensitive elements 31 is not hampered thereby.

FIG. 1C shows a third configuration in which the following are found from upstream to downstream of the detector 10: the protective sheet 23, the guide light 42, the scintillating substance 21 and the sensor 30. In this configuration, the exit surface of the light guide corresponds at least to its lower surface. Here, the flash of light emitted by the generator 40 leaves the guide 42 at its lower surface, passes through the scintillating substance 21 and arrives at the photosensitive elements 31. Like the second configuration shown in FIG. 1B, the reflection member 43 is not necessary in this configuration. So as to propagate along the light guide 42, the flash of light may be reflected at the interface between the light guide 42 and the protective sheet 23. Advantageously, in this configuration as well, the diffusion of light to the photosensitive elements 31 is not hampered thereby.

Notably, the light guide 42 is notably in contact with and in particular adhesively bonded to the scintillating substance 21 via its lower surface.

FIGS. 1D and 1E respectively show a fourth and a fifth configuration in which the light guide 42 is not an insert to the detector 10. In the fourth configuration, it is the substrate 32 of the sensor 30 that acts as light guide 42. In the fifth configuration, it is the support 22 that acts as light guide 42. These configurations are advantageous in that they make it possible to reduce the overall size of the light generator 40 and the production costs of the detector 10.

In the fourth configuration, the following are found from upstream to downstream of the detector 10: the support 22, the scintillating substance 21, the sensor 30 and a lower reflector 44. Here, the exit surface of the guide 42 corresponds at least to the upper surface of the substrate 32. In this configuration, the flash of light emitted by the light source 41 leaves the substrate 32 via its upper surface, passes between the photosensitive elements 31 and is reflected off faces located upstream of the photosensitive sensor 30 before reaching the photosensitive elements 31 via their upstream face, like the first configuration. More precisely, the flash of light may be reflected at the interface between the support 22 and the scintillating substance and/or at the interface between the scintillator screen 20 and the photosensitive sensor 30 off faces where there is a break in refractive index. The support 22 is notably made of an opaque material. So as to propagate along the light guide 42, the flash of light is reflected against the lower reflector 44 and may also be reflected off the rear face of the photosensitive elements 31. Thus, in this configuration, the rear surface of the photosensitive elements 31 may also be able to reflect visible light.

In the configuration that is shown, the substrate 32 should be transparent or translucent so as to let through light emitted by the light source 41 of the generator 40.

In the fifth configuration, the following are found from upstream to downstream of the detector 10: an upper reflector 45, the support 22, the scintillating substance 21 and the sensor 30. Here, the exit surface of the guide 42 corresponds at least to the lower surface of the support 22. The flash of light emitted by the light source 41 thus arrives directly at the photosensitive elements 31 via the lower surface of the scintillating substance 21. So as to propagate along the light guide 42, the flash of light may be reflected at the interface between the upper reflector 45 and the support 22. Advantageously, in this configuration, the diffusion of light to the photosensitive element 31 is not hampered thereby.

In the configuration that is shown, the support 22 should be transparent or translucent so as to let through light emitted by the light source 41 of the generator 40.

Reference will now be made to FIGS. 2A-4B, which show three configurations of the light generator 40 with reference to the first configuration shown in FIG. 1A. Nevertheless, a person skilled in the art will know how to easily adapt the configurations of the light generator 40 shown here to the configurations illustrated in FIGS. 1B to 1E in relation to the light guide 42. For each configuration of the light guide 42 illustrated in FIGS. 2A-4B, there is shown an embodiment comprising a single light source 41 arranged at the periphery of a side face of the light guide 42 (A), and also an embodiment comprising two light sources 41 arranged at the periphery of two opposing side faces of the light guide 42 (B).

FIGS. 2A and 2B illustrate a first configuration in which the one or more light sources 41 are in contact with a side face of the guide 42 and the light guide 42 has an upper and lower surface of the same dimension as that of the substrate 32 and of the scintillator screen 20. The path of a photon of the light flux from a light source 41 is shown.

More precisely, in FIG. 2A, the light generator 40 comprises:
one or more light sources arranged in contact with a single side face of the light guide,
a light guide extending under the entire surface of the substrate 32 of the sensor 30 and comprising a texturing in the form of a plurality of screen-printed dots 48 on its lower surface,
a diffuser 49 arranged between the substrate 32 and the light guide 42 and comprising a texturing in the form of an indentation 50 on its lower surface,
a lower reflector 44 and
a lateral reflector 46 arranged against the side face of the guide 42 opposite that of the at least one light source 41, and extending along a side face of the diffuser 49, of the substrate 32 and of the scintillating substance 21.

In FIG. 2B, this configuration is repeated with the exception of the lateral reflector 46, which is replaced with one or more other light sources 41.

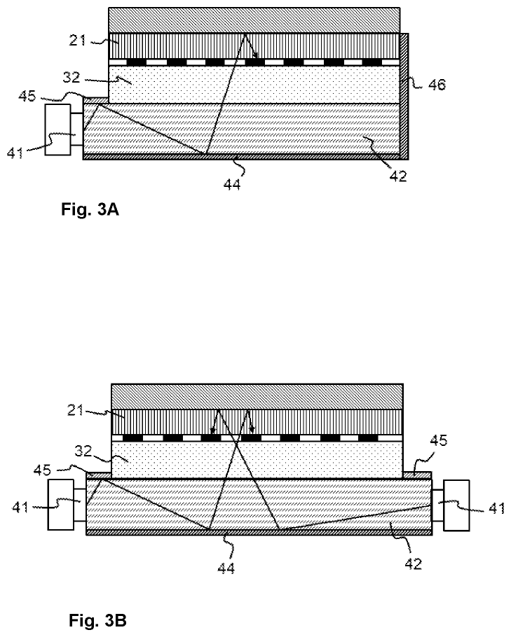

FIGS. 3A and 3B illustrate a second configuration in which the one or more light sources 41 are in contact with a side face of the guide 42 and the light guide has an upper and lower surface of dimensions greater than that of the substrate 32 and of the scintillator screen 20. The light guide thus has one or two offset lateral parts. The path of a photon of the light flux from a light source 41 is shown.

The embodiment shown in FIG. 3A comprises:
one or more light sources arranged in contact with a single side face of the offset lateral part of the light guide 42,
a light guide 42 having an upper and lower surface of dimensions greater than those of the substrate 32, so as to comprise an offset lateral part,
a lower reflector 44,
an upper reflector 45 arranged against the upper surface of the offset lateral part of the light guide 42 and
a lateral reflector 46 arranged against the side face of the guide 42 opposite that of the at least one light source 41, and extending along a side face of the substrate 32 and of the scintillating substance 21.

In FIG. 3B, this configuration is repeated with the exception of the lateral reflector 46, which is replaced with one or more other light sources 41 arranged at a second offset lateral part opposite the first and covered by a second lateral reflector 46.

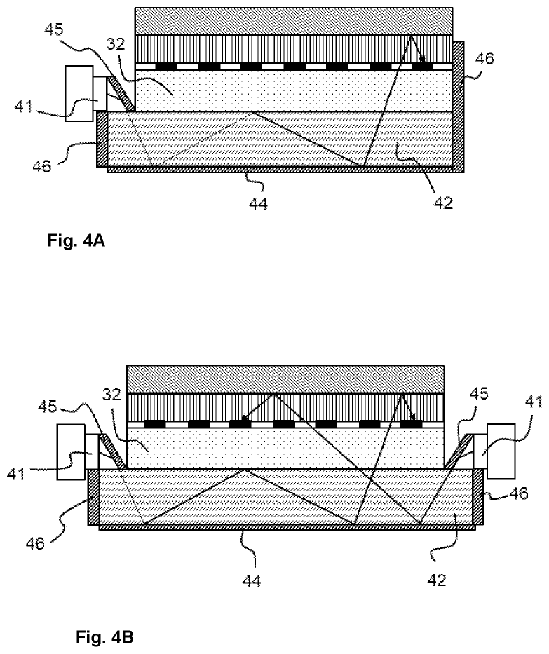

FIGS. 4A and 4B illustrate a third configuration in which the one or more light sources 41 are arranged above a side face of the guide 42, wherein the light guide 42 has an upper and lower surface of dimension greater than that of the substrate 32 and of the scintillator screen 20, wherein a lateral reflector 46 is arranged on each side face of the guide 42 and wherein an oblique upper reflector 45 is present so as to direct light from said sources 41 towards the inside of the guide 42. The path of a photon of the light flux from a light source 41 is shown.

1. A solid-state digital detector for detecting incident radiation, the detector comprising a photosensitive sensor and a light generator, the photosensitive sensor comprising photosensitive elements organized in a matrix, the light generator being intended to optically erase the photosensitive elements, wherein the light generator comprises:
a light guide comprising a front face facing the front of the detector, a rear face opposite the front face and at least one side face extending between the front face and the rear face, and
at least one light source arranged at the lateral periphery of the light guide, and wherein the light generator is configured such that said at least one light source injects light through at least one of the faces of the light guide,
said light guide being configured to distribute light from said at least one light source over the entire matrix of photosensitive elements.

2. The digital detector according to claim 1, wherein the light guide comprises a diffusive texturing in order to achieve a uniform distribution of light across the matrix of photosensitive elements.

3. The light detector according to claim 1, wherein the light guide comprises a set of microbubbles expediently arranged inside it in order to achieve a uniform distribution of light across the matrix of photosensitive elements.

4. The digital detector according to claim 1, wherein the photosensitive sensor comprises a transparent or translucent substrate on which the matrix of photosensitive elements is arranged and wherein the front face of the light guide is arranged against said substrate.

5. The digital detector according to claim 4, wherein the light guide is attached to the substrate of the photosensitive sensor, preferably by adhesive bonding.

6. The digital detector according to claim 1, wherein the photosensitive sensor comprises a transparent or translucent substrate on which the matrix of photosensitive elements is arranged and wherein said substrate forms the light guide.

7. The digital detector according to claim 1, wherein said at least one light source is arranged facing at least one of the side faces of the light guide.

8. The digital detector according to claim 1, wherein the light generator comprises at least one reflection member able to deflect light from said at least one light source towards the light guide.

9. The digital detector according to claim 7, wherein the reflection member comprises at least one dioptre and/or at least one reflector arranged facing one of the faces of the light guide.

10. The digital detector according to claim 1, wherein the light guide comprises at least two mutually opposing side faces, wherein the light generator comprises at least one lateral reflector arranged against at least one of the side faces of the light guide, and said at least one light source is preferably arranged facing the side face opposite that of the lateral reflector.

11. The digital detector according to claim 1, wherein the matrix of photosensitive elements is arranged on a transparent or translucent substrate, wherein the light guide is arranged downstream of said substrate and comprises a lateral part offset from that of said substrate, wherein the light generator comprises at least one upper reflector extending against all or part of the upper face of said at least one offset lateral part of the light guide, and wherein said at least one light source is arranged at the periphery of said at least one offset lateral part of the light guide.
